(19) United States
(12) Patent Application Publication
(10) Pub. No.: US 2011/0104234 A1
(43) Pub. Date: May 5, 2011

(54) Biodegradable Modified Carpolactone Polymers for Fabrication and Coating Medical Devices

(75) Inventors: Mingfei Chen, Santa Rosa, CA (US); Peiwen Cheng, Santa Rosa, CA (US); Ya Guo, Rohnert Park, CA (US); Kishore Udipi, Santa Rosa, CA (US)

(73) Assignee: Medtronic Vascular, Inc., Santa Rosa, CA (US)

(21) Appl. No.: 12/975,094

(22) Filed: Dec. 21, 2010

Related U.S. Application Data
(62) Division of application No. 11/383,262, filed on May 15, 2006, now abandoned.

(51) Int. Cl. A61K 9/00; A61F 2/82; A61K 39/395; A61K 31/7088; A61K 33/00; A61P 9/10

(52) U.S. Cl. 424/426; 623/1.46; 424/422; 424/130.1; 514/44 A; 514/44 R; 424/718

(57) ABSTRACT

Disclosed herein are biodegradable modified caprolactone polymers for coating and forming medical devices. The properties of the polymers are fine tuned for optimal performance depending on the medical purpose. Moreover, the polymers are suitable for the controlled in situ release of drugs at the treatment site.

### FIELD OF THE INVENTION

The invention disclosed herein relates to modified caprolactone monomers for the synthesis of biodegradable polymers. Moreover, the biodegradable polymers are for forming and coating implantable medical devices and controlling in situ drug release.

### BACKGROUND OF THE INVENTION

Cardiovascular disease, specifically atherosclerosis, remains a leading cause of death in developed countries. Atherosclerosis is a multifactorial disease that results in a narrowing, or stenosis, of a vessel lumen. Briefly, pathologic inflammatory responses resulting from vascular endothelium injury causes monocytes and vascular smooth muscle cells (VSMCs) to migrate from the sub endothelium and into the arterial wall's intimal layer. There the VSMC proliferate and lay down an extracellular matrix causing vascular wall thickening and reduced vessel patency.

Cardiovascular disease caused by stenotic coronary arteries is commonly treated using either coronary artery by-pass graft (CABG) surgery or angioplasty. Angioplasty is a percutaneous procedure wherein a balloon catheter is inserted into the coronary artery and advanced until the vascular stenosis is reached. The balloon is then inflated restoring arterial patency. One angioplasty variation includes arterial stent deployment. Briefly, after arterial patency has been restored, the balloon is deflated and a vascular stent is inserted into the vessel lumen at the stenosis site. The catheter is then removed from the coronary artery and the deployed stent remains implanted to prevent the newly opened artery from constricting spontaneously. However, balloon catheterization and stent deployment can result in vascular injury ultimately leading to VSMC proliferation and neointimal formation within the previously opened artery. This biological process whereby a previously opened artery becomes re-occluded is referred to as restenosis.

The introduction of intracoronary stents into clinical practice has dramatically changed treatment of obstructive coronary artery disease. Since having been shown to significantly reduce restenosis as compared to percutaneous transluminal coronary angioplasty (PTCA) in selected lesions, the indication for stent implantation was been widened substantially. As a result of a dramatic increase in implantation numbers worldwide in less selected and more complex lesions, in-stent restenosis (ISR) has been identified as a new medical problem with significant clinical and socioeconomic implications. The number of ISR cases is growing: from 100,000 patients treated worldwide in 1997 to an estimated 150,000 cases in 2001 in the United States alone. ISR is due to a vascular response to injury, and this response begins with endothelial denudation and culminates in vascular remodeling after a significant phase of smooth muscle cell proliferation.

Additionally, recent advances in in situ drug delivery have led to the development of implantable medical devices specifically designed to provide therapeutic compositions to remote anatomical locations. Perhaps one of the most exciting areas of in situ drug delivery is in the field of intervention cardiology. Vascular occlusions leading to ischemic heart disease are frequently treated using percutaneous transluminal coronary angioplasty (PTCA) whereby a dilation catheter is inserted through a femoral artery incision and directed to the site of the vascular occlusion. The catheter is dilated and the expanding catheter tip (the balloon) opens the occluded artery restoring vascular patency. Generally, a vascular stent is deployed at the treatment site to minimize vascular recoil and restenosis. However, in some cases stent deployment leads to damage to the intimal lining of the artery which may result in vascular smooth muscle cell hyperproliferation and restenosis. When restenosis occurs it is necessary to either re-dilate the artery at the treatment site, or, if that is not possible, a surgical coronary artery bypass procedure must be performed.

Generally, implantable medical devices are intended to serve long term therapeutic applications and are not removed once implanted. In some cases it may be desirable to use implantable medical devices for short term therapies. However, their removal may require highly invasive surgical procedures that place the patient at risk for life threatening complications. Therefore, it would be desirable to have medical devices designed for short term applications that degrade via normal metabolic pathways and are reabsorbed into the surrounding tissues.

In general, polymer selection criteria for use as biomaterials are to match the mechanical properties of the polymer(s) and degradation time to the needs of the specific in vivo application. The factors affecting the mechanical performance of biodegradable polymers are those that are well known to the polymer scientist, and include monomer selection, initiator selection, process conditions and the presence of additives. These factors in turn influence the polymer's hydrophilicity, crystallinity, melt and glass-transition temperatures, molecular weight, molecular-weight distribution, end groups, sequence distribution (random versus blocky) and presence of residual monomer or additives. In addition, the polymer scientist working with biodegradable materials must evaluate each of these variables for its effect on biodegradation. Known biodegradable polymers include, among others, polyglycolide (PGA), polylactide (PLA) and poly(c-caprolactone) (PCA). However, these polymers are generally hydrophobic and their structures are difficult to modify. Consequently, the polymer's physical characteristics are difficult to modify, or tune, to match specific clinical demands. For example, polymers made from PLA are extremely slow to degrade and thus not suited for all applications. To address this deficiency polymer scientists have developed co-polymers of PLA and PCA. However, biodegradation rates remain significantly limited.

Implanted medical devices that are coated with biodegradable biocompatible polymers offer substantial benefits to the patient. Reduced inflammation and immunological responses promote faster post-implantation healing times in contrast to uncoated medical devices. Polymer-coated vascular stents, for example, may encourage endothelial cell proliferation and therefore integration of the stent into the vessel wall. Loading the coating polymers with appropriate drugs is also advantageous in preventing undesired biological responses. For example, an implanted polylactic acid polymer loaded with hirudin and prostacyclin does not generate thrombosis, a major cause of post-surgical complications (Eckhard et al, Circulation, 2000, pp 1453-1458).

There is a need for improved polymeric materials suitable for forming or coating implantable medical devices. The implantable polymeric materials should be able to deliver hydrophilic and hydrophobic drugs, effectively coat the medical device and be biodegradable. The present invention addresses these problems by providing polymers comprising that are biocompatible, biodegradable and suitable for forming and coating implantable medical devices.

### SUMMARY OF THE INVENTION

The present invention relates to biodegradable biocompatible polymers comprising modified caprolactone monomers that are suitable for forming and coating implantable medical devices as well as controlling in situ drug release. The polymers of the present invention have polyester and polyether backbones and are comprised of monomers including, but not limited to, ε-caprolactone, 1,8 octanediol, polyethylene glycol (PEG), trimethylene carbonate, lactide, glycolide, modified caprolactone monomers and their derivatives. Structural integrity and mechanical durability are provided through the use of monomers including lactide and glycolide. Elasticity is provided by monomers including caprolactone and trimethylene carbonate. The polymers of the present invention are capable of delivering both hydrophobic and hydrophilic drugs to a treatment site. Furthermore, the polymers of the present invention are biodegradable. Varying the monomer ratios allows the practitioner to fine tune, or modify, the properties of the polymer to control physical properties including drug elution rates.

The properties of biodegradable biocompatible polymers are a result of the monomers used and the reaction conditions employed in their synthesis including, but not limited to, temperature, solvent choice, reaction time and catalyst choice.

The polymers made in accordance with the present invention are also suitable for manufacturing implantable medical devices. In one embodiment of the present invention, a medical device is manufactured from a biodegradable biocompatible polymer of the present invention. In another embodiment, the biodegradable biocompatible polymer is provided as a coating on a medical device. In yet another embodiment, a drug is provided in the biodegradable biocompatible polymer medical device or coating.

Medical devices suitable for coating with the polymers of the present invention include, but are not limited to, vascular stents, stent grafts, urethral stents, bile duct stents, catheters, guide wires, pacemaker leads, bone screws, sutures and prosthetic heart valves. The polymers of the present invention are suitable for coating and manufacturing implantable medical devices. Medical devices which can be manufactured from the polymers of the present invention include, but are not limited to, vascular stents, stent grafts, urethral stents, bile duct stents, catheters, guide wires, pacemaker leads, bone screws, sutures and prosthetic heart valves.

The present invention also provides biodegradable biocompatible polymer with variable properties that include glass transition temperatures (Tg). Drug elution from polymers depends on many factors including polymer density. The drug to be eluted, molecular nature of the polymer and Tg, among other properties. Higher Tgs, for example temperatures above 40° C., result in more brittle polymers while lower Tgs, e.g lower than 40° C., result in more pliable and elastic polymers. In the present invention Tg can be controlled, such that the polymer elasticity and pliability can be varied as a function of temperature. The mechanical properties dictate the use of the polymers, for example, drug elution is slow from polymers that have high Tgs while faster rates of drug elution are observed with polymers possessing low Tgs.

### DEFINITION OF TERMS

Prior to setting forth the invention, it may be helpful to an understanding thereof to set forth definitions of certain terms that will be used hereinafter:

1, 4 addition reaction: As described herein, 1, 4 addition is the addition of a nucleophile to a α, β unsaturated carbonyl compound at the terminal alkene (Reaction 1). The example presented in Reaction 1 is non-limiting.

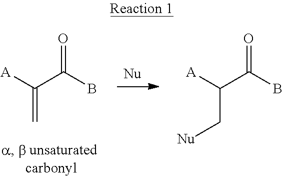

Lactone: As used herein “lactone” or “lactone ring” refers to a cyclic ester. It is the condensation product of an alcohol group and a carboxylic acid group in the same molecule. Prefixes may indicate the ring size: beta-lactone (4-membered), gamma-lactone (5-membered), delta-lactone (6-membered ring).

Lactide: As used herein, lactide refers to 3,6-dimethyl-1,4-dioxane. More commonly lactide is also referred to herein as the heterodimer of R and S forms of lactic acid, the homodimer of the S form of lactic acid and the homodimer of the R form of lactic acid. Lactide is also depicted below in Formula 1. Lactic acid and lactide are used interchangeably herein. The term dimer is well known to those ordinarily skilled in the art.

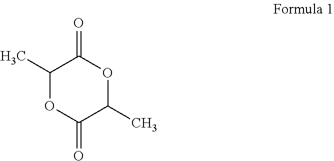

Glycolide: As used herein, glycolide refers to a chemical of the structure of Formula 2.

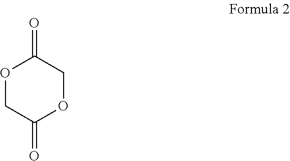

4-tert-butyl caprolactone: As used herein 4-tert-butyl caprolactone refers to a chemical of the structure of Formula 3.

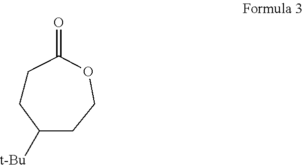

Amphiphilic: As used herein, amphiphilic refers to a molecule or polymer having at least one a polar, water-soluble group and at least one a nonpolar, water-insoluble group. In simpler non limiting terms, a molecule that is soluble in both an aqueous environment and a non-aqueous environment.

Backbone: As used here in “backbone” refers to the main chain of a polymer or copolymer of the present invention. A “polyester backbone” as used herein refers to the main chain of a biodegradable polymer comprising ester linkages. A “polyether backbone” as used herein refers to the main chain of a biodegradable polymer comprising ether linkages. An exemplary polyether is polyethylene glycol (PEG).

Biodegradable: As used herein “biodegradable” refers to a polymeric composition that is biocompatible and subject to being broken down in vivo through the action of normal biochemical pathways. From time to time bioresorbable and biodegradable may be used interchangeably, however they are not coextensive. Biodegradable polymers may or may not be reabsorbed into surrounding tissues, however all bioresorbable polymers are considered biodegradable. The biodegradable polymers of the present invention are capable of being cleaved into biocompatible byproducts through chemical- or enzyme-catalyzed hydrolysis.

Copolymer: As used here in a “copolymer” will be defined as a macromolecule produced by the simultaneous or step-wise polymerization of two or more dissimilar units such as monomers. Copolymer shall include bipolymers (two dissimilar units), terpolymers (three dissimilar units), etc.

Compatible: As used herein “compatible” refers to a composition possessing the optimum, or near optimum combination of physical, chemical, biological and drug release kinetic properties suitable for a controlled-release coating made in accordance with the teachings of the present invention. Physical characteristics include durability and elasticity/ductility, chemical characteristics include solubility and/or miscibility and biological characteristics include biocompatibility. The drug release kinetic should be either near zero-order or a combination of first and zero-order kinetics.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

Drug(s): As used herein “drug” shall include any bioactive agent having a therapeutic effect in an animal. Exemplary, non limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Ductility: As used herein “ductility, or ductile” is a polymer attribute characterized by the polymer's resistance to fracture or cracking when folded, stressed or strained at operating temperatures. When used in reference to the polymer coating compostions of the present invention the normal operating temperature for the coating will be between room temperature and body temperature or approximately between 15° C. and 40° C. Polymer durability in a defined environment is often a function of its elasticity/ductility.

Glass Transition Temperature (Tg): As used herein glass transition temperature (Tg) refers to a temperature wherein a polymer structurally transitions from a elastic pliable state to a rigid and brittle state.

Hydrophilic: As used herein in reference to the bioactive agent, the term “hydrophilic” refers to a bioactive agent that has a solubility in water of more than 200 micrograms per milliliter.

Hydrophobic: As used herein in reference to the bioactive agent the term “hydrophobic” refers to a bioactive agent that has a solubility in water of no more than 200 micrograms per milliliter.

N-acetyl caprolactone: As used herein N-acetyl caprolactone refers to a chemical of the structure of Formula 4.

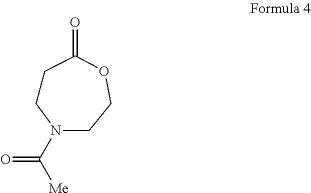

Modified caprolactone: As used herein modified caprolactone refers to derivatives of caprolactone, as depicted in Formula 5, such that carbons 1 through 5 have at least 1 atom bonded directly. In other terms modified caprolactone is defined as carbons 1 through 5 having at least 1 atom replacing a hydrogen atom of caprolactone.

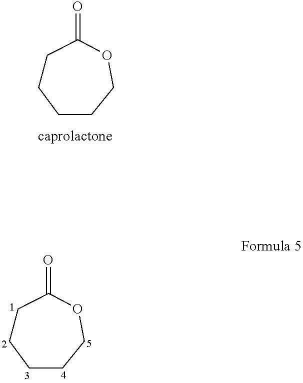

Mₙ: As used herein Mₙ refers to number-average molecular weight. Mathematically it is represented by the following formula: Mₙ=ΣᵢNᵢMᵢ/ΣᵢNᵢ, wherein the Nᵢ is the number of moles whose weight is M.

M_{w}: As used herein M_{w} refers to weight average molecular weight that is the average weight that a given polymer may have. Mathematically it is represented by the following formula: M_{W}=ΣᵢNᵢMᵢ²/ΣᵢMᵢ, wherein Nᵢ is the number of molecules whose weight is M.

### DETAILED DESCRIPTION OF THE INVENTION

The present invention relates to biodegradable biocompatible polymers comprising modified caprolactone monomers that are suitable for forming and coating medical devices as well as controlling in situ drug release. The polymers of the present invention have polyester and polyether backbones and are comprised of monomers including, but not limited to, ε-caprolactone, 1,8 octanediol, polyethylene glycol (PEG), trimethylene carbonate, lactide, glycolide, modified caprolactone monomers and their derivatives. Structural integrity and mechanical durability are provided with monomers including lactide and glycolide. Elasticity is provided by monomers including caprolactone and trimethylene carbonate. Therefore the polymers of the present invention are capable of delivering both hydrophobic and hydrophilic drugs to a treatment site. Furthermore, the polymers of the present invention are biodegradable. Varying the monomer ratios allows the practitioner to fine tune, or modify, the properties of the polymer to control physical properties including drug elution rates.

The properties of biodegradable biocompatible modified caprolactone polymers are a result of the monomers used and the reaction conditions employed in their synthesis including, but not limited to, temperature, solvent choice, reaction time and catalyst choice.

The polymers made in accordance with the present invention are also suitable for manufacturing implantable medical devices. In one embodiment of the present invention, a medical device is manufactured from a biodegradable biocompatible polymer of the present invention. In another embodiment, the biodegradable biocompatible polymer is provided as a coating on a medical device. In yet another embodiment, a drug is provided in the biodegradable biocompatible polymer medical device or coating.

Moreover, the polymers of the present invention are suitable for the deliverly drugs from an implantable medical device made wherein the polymer is coated on at least one surface of the medical device, thereby allowing for controlled drug release directly to the implantation site. Hydrophobic polymers including polylactic acid, polyglycolic acid and polycaprolactone are generally compatible with hydrophobic drugs. Hydrophilic polymers conversely are more compatible with hydrophilic drugs. Polymer-drug incompatibility hurdles are overcome by using modified caprolactone polymers which are amphiphilic. In one example, biodegradable modified caprolactone polymers are provided with hydrophilic groups containing poly-ionic organic moieties and the hydrophobic portion of the polymer contains a steroid, e.g. cholesterol coupled to a poly-lactide (see U.S. Pat. No. 5,932,539).

Medical devices suitable for coating with the polymers of the present invention include, but are not limited to, vascular stents, stent grafts, urethral stents, bile duct stents, catheters, guide wires, pacemaker leads, bone screws, sutures and prosthetic heart valves. The polymers of the present invention are suitable for coating and manufacturing implantable medical devices. Medical devices which can be manufactured from the polymers of the present invention include, but are not limited to, vascular stents, stent grafts, urethral stents, bile duct stents, catheters, guide wires, pacemaker leads, bone screws, sutures and prosthetic heart valves.

The present invention also provides for biodegradable biocompatible polymers with variable properties that include glass transition temperatures (Tg). Drug elution from polymers depends on many factors including polymer density, the drug to be eluted, molecular nature of the polymer and Tg, among other properties. Higher Tgs, for example temperatures above 40° C., result in more brittle polymers while lower Tgs, e.g lower than 40° C., result in more pliable and elastic polymers. In the present invention Tg can be controlled such that the polymer elasticity and pliability can be varied as a function of temperature. The mechanical properties dictate the use of the polymers, for example, drug elution is slow from polymers that have high Tgs while faster rates of drug elution are observed with polymers possessing low Tgs.

The present invention provides for polymers that incorporate modified caprolactone monomers. The polymers of the present invention include monomers further comprising diols. In one embodiment of the present invention the diol-containing monomer is 1,8 octanediol (CAS#629-41-4). In yet another embodiment of the present invention the diol-containing monomer is PEG.

The modified caprolactone monomers that comprise the polymers of the present invention include 4-tert-butyl caprolactone and N-acetyl caprolactone. The modified caprolactone monomers are synthesized by a variety of synthetic methods including oxidation of ketones with hydroperoxides, known to those of ordinary skill in the art as Baeyer-Villiger reactions. Typically the oxidations are conducted with meta-chloroperbenzoic acid (3-chloroperbenzoic acid, CAS#937-14-4) or mCPBA and yield esters or lactones. An exemplary, non-limiting Baeyer-Villiger reaction involving a general hydroperxide and a general ketone providing a general ester is shown in Reaction 2.

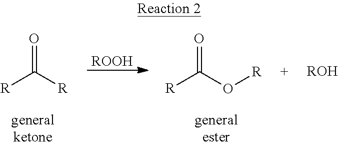

Conducting Baeyer-Villiger reactions on cyclohexanones results in the formation of caprolactone. Further, conducting Baeyer-Villiger reactions on cyclohexanone derivatives yields modified caprolactone monomers. Exemplary cyclohexanone derivatives include 4-tert-butyl cyclohexanone, 2-decalone, 1-decalone, 2-methyl cyclohexanone, 3-methyl cyclohexanone, 4-methyl cyclohexanone and other moieties. Cyclohexanone derivatives suitable for forming modified caprolacone monomers for the polymers of the present invention is depicted in Formula 6, wherein R₁, R₂, R₃, and R₄ individually are moieties including, but not limited to, methyl, ethyl, hydrogen, linear and branched chains with C₁ to C₁₈, cyclic moieties having C₃ to C₈ including, but not limited to, heterocycles of nitrogen, oxygen and sulfur and combinations thereof.

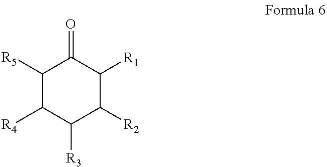

A cyclohexanone derivative suitable for forming modified caprolactone monomers for the polymers of the present invention is a compound in which cycling rings are fused to the cyclohexanone skeletal structure, for example 2-decalone (CAS# 4832-17-1). In the case of 2-decalone, the cyclohexane ring is fused to a cyclohexanone having the structure of Formula 6 from R₂ to R₃ or R₄ to R₃. In an embodiment of the present invention, modified caprolactone monomers synthesized from cyclohexanone derivatives include cyclic rings that are fused on the cyclohexanone, wherein the cyclic rings are C₃ to C₈ including, but not limited to, heterocycles of nitrogen, oxygen and sulfur and combinations thereof.

The modified caprolactone monomers are also synthesized from the general caprolactone of Formula 5 by other common synthetic methods.

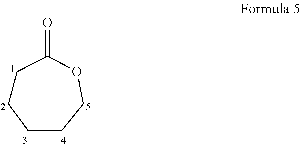

For example, alkylation of caprolactone with enolate chemistry on carbon 1 of the caprolactone of Formula 5 is a facile process known to those of ordinary skill in the art. In another non limiting method, 1, 4 addition reactions can be employed to alkylate carbon 2 of the caprolactone of Formula 5. For example, Formula 7 undergoes a 1, 4 reaction with a nucelophile (Nu) to produce a modified caprolactone of Formula 8 wherein carbon 2 is now substituted. Suitable nucleophiles include, but are not limited to, amines, phosphorous compounds, alkyl groups, aryl groups, alkenyl groups and alkynyl groups.

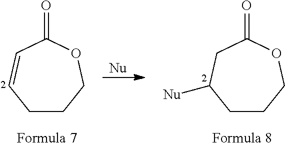

Producing modified caprolactone monomers for polymers of the present invention may also include substitutions at carbon 3 of the caprolactone of Formula 5. Widely available 4-substituted cyclohexanones, such as the exemplary molecule of Formula 9, are commercially available. In Formula 9, R comprises either a branched or linear alkyl, alkenyl or alkynyl chain from C₁ to C₁₈. In cyclic forms, R further comprises rings of C₃ to C₈ including but not limited to heterocycles of nitrogen, oxygen and sulfur and combinations thereof. As depicted below, Baeyer-Villiger reactions can be employed in the synthesis of modified caprolactone monomers by substitution at carbon 3 to form Formula 10. In one embodiment of the present invention, R is tert-butyl and the modified caprolactone monomer produced is 4-tert-butyl caprolactone (Formula 10 wherein R is tert-butyl).

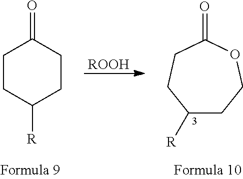

Producing modified caprolactone monomers for polymers of the present invention may also include substitutions at carbon 4 of the caprolactone of Formula 5. Substituted cyclohexanones of Formula 11 are widely available commercially and also synthesized by those of ordinary skill in the art with facile methods from cyclohexenone (CAS#930-68-7). Deprotonation of the a proton of Formula 11 with a hindered base (HB) will result in an enolate opposite of the R group. Trapping the enolate with Me₃SiCl then yields the silyl enol ether depicted in Formula 12. Manipulating the double bond to yield Formula 13 is accomplished by the Saegusa-Ito reaction (J. Org. Chem. 1978. 43(5), 1011-1013). A general Baeyer-Villiger reaction then forms the caprolactone of Formula 14. Finally, hydrogenation of Formula 14 with hydrogen gas and palladium on carbon yields the modified caprolactone monomer of Formula 15. In Formula 15, R comprises either a branched or linear alkyl, alkenyl or alkynyl chain from C₁ to C₁₈. In cyclic forms, R further comprises rings of C₃ to C₈ including, but not limited to, heterocycles of nitrogen, oxygen and sulfur and combinations thereof.

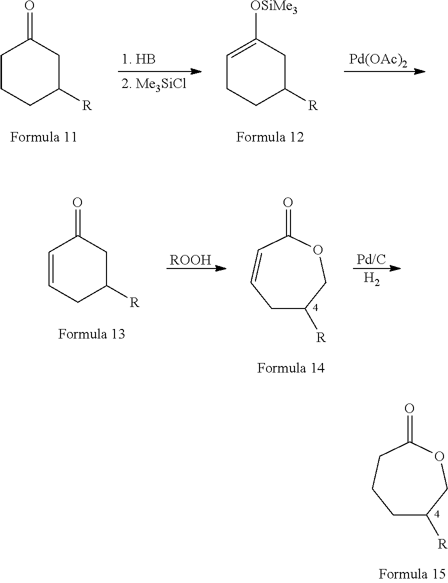

Producing modified caprolactone monomers for polymers of the present invention may also include substitutions at carbon 5 of the caprolactone of Formula 5. Starting from the general diol depicted in Formula 16, a TPAP (tetrapropylammonium perruthenate, CAS#114615-82-6)/NMO (N-methyl morpholine oxide, CAS#7529-22-8) oxidation will yield the modified caprolactone monomer of Formula 17. In Formula 17, R comprises either a branched or linear alkyl, alkenyl or alkynyl chain from C₁ to C₁₈. In cyclic forms, R further comprises rings of C₃ to C₈ including, but not limited to, heterocycles of nitrogen, oxygen and sulfur and combinations thereof.

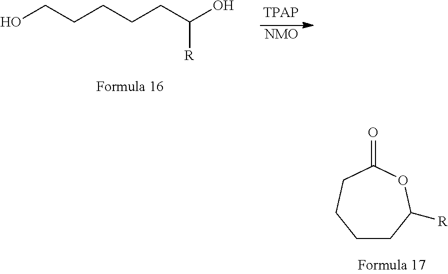

The modified caprolactone monomers may be substituted at more than one carbon. In one embodiment of the present invention, the modified caprolactone monomers are substituted on at least two carbons of caprolactone, for example and not intended as a limitation, as in Formula 18:

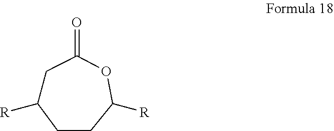

The biodegradable modified caprolactone polymers of the present invention comprise modified caprolactone monomers. Polymers of the present invention include copolymers comprising at least two monomers. In one embodiment, the polymers of the present invention comprise monomers including ε-caprolactone, trimethylene carbonate, lactide, glycolide, modified caprolactone monomers, 1,8 octanediol and their derivatives. In another embodiment of the present invention, the biodegradable modified caprolactone polymer comprises 4-tert-butyl caprolactone and lactide. In still another embodiment of the present invention, the biodegradable modified caprolactone polymer comprises 4-tert-butyl caprolactone and glycolide. In yet another embodiment of the present invention, the biodegradable modified caprolactone polymer comprises 4-tert-butyl caprolactone, glycolide and lactide.

In one embodiment, the polymers of the present invention can be used to fabricate and coat medical devices. Coating polymers having relatively high Tgs can result in medical devices with unsuitable drug eluting properties as well as unwanted brittleness. In the cases of polymer-coated vascular stents, a relatively low Tg in the coating polymer effects the deployment of the vascular stent. For example, polymer coatings with low Tgs are “sticky” and adhere to the balloon used to expand the vascular stent during deployment, causing problems with the deployment of the stent. Low Tg polymers, however, have beneficial features in that polymers having low Tgs are more elastic at a given temperature than polymers having higher Tgs. Expanding and contracting a polymer-coated vascular stent mechanically stresses the coating. If the coating is too brittle, i.e. has a relatively high Tg, then fractures may result in the coating possibly rendering the coating inoperable. If the coating is elastic, i.e has a relatively low Tg, then the stresses experienced by the coating are less likely to mechanically alter the structural integrity of the coating. Therefore, the Tgs of the polymers of the present invention can be fine tuned for appropriate coating applications by a combination of monomer composition and synthesis conditions. The polymers of the present invention are engineered to have adjustable physical properties enabling the practitioner to choose the appropriate polymer for the function desired.

In order to tune, or modify, the polymers of the present invention, a variety of properties are considered including, but not limited to, Tg, connectivity, molecular weight and thermal properties.

In the present invention, the balance between the hydrophobic and hydrophilic properties in the biodegradable modified caprolactone polymer is controlled. Drug-eluting properties of the biodegradable modified caprolactone polymers can be tailored to a wide range of drugs. For example, increasing the hydrophobic nature of the polymer increases the polymer's compatibility with hydrophobic drugs. In the case where medical devices coated with polymers of the present invention is desired, the polymers can be tailored to adhere to the particular medical device.

The polymers of the present invention, therefore, can be used to form and to coat implantable medical devices. The polymers of the present invention are also useful for the delivery and controlled release of drugs. Drugs that are suitable for release from the polymers of the present invention include, but are not limited to, anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

In one embodiment of the present invention, the drug is covalently bonded to a modified caprolactone polymer of the present invention. The covalently-bound drug is released in situ from the biodegrading polymer with the polymer degradation products thereby ensuring a controlled drug supply throughout the degradation course. The drug is released to the treatment site as the polymeric material is exposed through biodegradation.

In another embodiment, the drug is dispersed in the polymer and released at the treatment site upon degradation.

Coating implantable medical devices with biodegradable modified caprolactone polymers that also control drug release is therapeutically advantageous to the patient. Post surgical complications involving medical device implants, e.g. vascular stents, are frequent. Administering drugs combating thrombosis, for example, is a common practice after surgical procedures, especially after cardiothoracic interventions. Drug releasing polymeric coatings on implanted medical devices can offset post surgical side effects by delivering therapeutic agents, such as drugs, directly to the affected areas.

Implantable medical devices suitable for coating with the biodegradable modified caprolactone polymers of the present invention include, but are not limited to, vascular stents, stent grafts, urethral stents, bile duct stents, catheters, guide wires, pacemaker leads, bone screws, sutures and prosthetic heart valves. The polymers of the present invention are suitable for coating and manufacturing implantable medical devices. Medical devices which can be manufactured from the biodegradable modified caprolactone polymers of the present invention include, but are not limited to, vascular stents, stent grafts, urethral stents, bile duct stents, catheters, guide wires, pacemaker leads, bone screws, sutures and prosthetic heart valves.

The controlled release modified caprolactone polymer coatings of the present invention can be applied to medical device surfaces, either primed or bare, in any manner known to those skilled in the art. Applications methods compatible with the present invention include, but are not limited to, spray coating, electrostatic spray coating, plasma coating, dip coating, spin coating and electrochemical coating.

The methods described are also useful for coating only a portion of the implantable medical device such that the medical device contains portions that provide the beneficial effects of the coating and portions that are uncoated. The coating steps can be repeated or the methods combined to provide a plurality of layers of the same coating or a different coating. In one embodiment, each layer of coating comprises a different polymer or the same polymer. In another embodiment each layer comprises the same drug or a different drug.

In one embodiment of the present invention, a modified caprolactone polymer of the present invention is chosen for a particular use based upon its physical properties. For example, a polymer coating provides additional structural support to a medical device by increasing the content of lactic acid in the polymer. In still another embodiment, a polymer coating on a medical device decreases friction between the medical device and the surrounding tissue, or between the medical device and the delivery system, facilitating the implantation procedure.

The biodegradable modified caprolactone polymers of the present invention are particularly suitable for manufacturing implantable medical devices since the methods and compositions disclosed herein allow the fine tuning of the structural properties of the polymers by using various ratios of monomers in the synthesis of the polymers. One such property is degradation time. The biodegradable modified caprolactone polymers described herein can be tuned to biodegrade at various lengths of time by varying the monomer composition of the polymer.

In one embodiment of the present invention, a vascular stent is manufactured from the biodegradable modified caprolactone polymers of the present invention. The advantages of the biodegradable modified caprolactone polymer coating also apply to vascular stents manufactured from biodegradable modified caprolactone polymers.

### EXAMPLES

The following non limiting examples provide methods for the synthesis of exemplary monomers, polymers and medical devices according to the teachings of the present invention.

### Example 1

In Example 1 the synthesis of a modified caprolactone monomer is described, specifically 4-tert-butyl caprolactone.

To a cooled (0° C.) solution of 4-tert-butyl cyclohexanone (50.0 g, 0.324 mol) in dichloromethane (100 mL) is slowly added 3-chloroperbenzoic acid (90.0 g, 0.365 mol, purity of 70%) in dichloromethane (450 mL). After the reaction is complete the mixture is filtered and is first washed with sodium thiosulfate (15% wt/v, 2×200 mL) and second with sodium bicarbonate (saturated, 5×200 mL). The organic solution is dried with sodium sulfate and filtered. The solvent is removed in vacuo and resulting material purified by vacuum distillation (collected: 118-124° C. at 0.08 torr) to yield a solid material (70%) with a melting point range of 49-51° C.

### Example 2

In Example 2 the synthesis of modified caprolactone copolymers is described, specifically copolymers comprising 4-tert-butyl caprolactone and lactide. A general procedure follows.

To a mixture of tin octoate, 4-tert-butyl caprolactone is added 1,8 octanediol and lactide. The atmosphere of the reaction chamber is subjected to five vacuum/argon cycles. The mixture is then heated (125° C.) for 72 hours. The resulting polymers are precipitated from methanol and chloroform solutions.

TABLE 1
Formulations for Example 2.

| | Tin Octoate | 1,8 Octanediol | 4-tert-butyl | Lactide |
|---|---|---|---|---|
| Polymer No. | (g) | (g) | caprolactone (g) | (g) |
| 1 | 0.0203 | 0.0061 | 1.6000 | 6.4000 |
| 2 | 0.0192 | 0.0068 | 3.2000 | 4.8000 |
| 3 | 0.0208 | 0.0060 | 4.8000 | 3.2000 |
| 4 | 0.0190 | 0.0060 | 6.4000 | 1.6000 |

TABLE 2
Properties of Formulations for Example 2.

| | 4-tert-butyl | | | |
|---|---|---|---|---|
| Polymer | caprolactone | | | Tg |
| No. | (mol %) | Mₙ | M_{w} | (° C.) |
| 1 | 8.05 | 106947 | 173212 | 35.9 |
| 2 | 16.8 | 181782 | 376189 | 25.2 |
| 3 | 36.4 | 116704 | 220060 | 12.3 |
| 4 | 61.5 | 81120 | 172322 | 3.9 |

### Example 3

In Example 3 the synthesis of a modified caprolactone monomer is described, specifically a cyclohexyl fused caprolactone.

To a cooled (0° C.) solution of 2-decalone (50.0 g, 0.328 mol) in dichloromethane (100 mL) is slowly added 3-chloroperbenzoic acid (90.0 g, 0.365 mol, purity of 70%) in dichloromethane (450 mL). After the reaction is complete the mixture is filtered and is first washed with sodium thiosulfate (15% wt/v, 2×200 mL) and second with sodium bicarbonate (saturated, 5×200 mL). The organic solution is dried with sodium sulfate and filtered. The solvent is removed in vacuo to present the cyclohexyl caprolactone.

### Example 4

The present invention pertains to biodegradable modified caprolactone polymers used for the manufacture of medical devices and medical devices coatings. The biodegradable modified caprolactone polymers disclosed in the present invention retain and release bioactive drugs. Example 3 discloses a non-limiting method for manufacturing stents made of biodegradable modified caprolactone polymers according to the teachings of the present invention.

For exemplary, non-limiting, purposes a vascular stent will be described. A biodegradable modified caprolactone polymer is heated until molten in the barrel of an injection molding machine and forced into a stent mold under pressure. After the molded polymer (which now resembles and is a stent) is cooled and solidified the stent is removed from the mold. In one embodiment of the present invention the stent is a tubular shaped member having first and second ends and a walled surface disposed between the first and second ends. The walls are composed of extruded polymer monofilaments woven into a braid-like embodiment. In the second embodiment, the stent is injection molded or extruded. Fenestrations are molded, laser cut, die cut, or machined in the wall of the tube. In the braided stent embodiment monofilaments are fabricated from polymer materials that have been pelletized then dried. The dried polymer pellets are then extruded forming a coarse monofilament which is quenched. The extruded, quenched, crude monofilament is then drawn into a final monofilament with an average diameter from approximately 0.01 mm to 0.6 mm, preferably between approximately 0.05 mm and 0.15 mm. Approximately 10 to approximately 50 of the final monofilaments are then woven in a plaited fashion with a braid angle about 90 to 170 degrees on a braid mandrel sized appropriately for the application. The plaited stent is then removed from the braid mandrel and disposed onto an annealing mandrel having an outer diameter of equal to or less than the braid mandrel diameter and annealed at a temperature between about the polymer glass transition temperature and the melting temperature of the polymer blend for a time period between about five minutes and about 18 hours in air, an inert atmosphere or under vacuum. The stent is then allowed to cool and is then cut.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1-14. (canceled)

15. An implantable medical device comprising a biodegradable polymer and a drug contained therein, wherein the biodegradable polymer comprises:
a first monomer comprising a modified caprolactone comprising a caprolactone of Formula 5 substituted at least one of carbons 1 through 5: 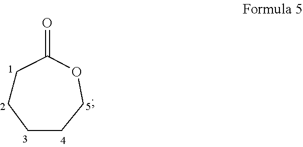
and
a second monomer selected from the group consisting of lactide, glycolide, trimethyene carbonate, 1,8 octanediol, polyethylene glycol, ε-caprolactone, and combinations thereof;
wherein the biodegradable polymer has a Tg of less than 40° C.

16. (canceled)

17. The implantable medical device of claim 15 wherein said modified caprolactone is substituted at carbon 1 of Formula 5 and said substituent group comprises a branched or linear alkyl, alkenyl or alkynyl chain from C₁ to C₁₈, cyclic moieties having C₃ to C₈ including heterocycles of nitrogen, oxygen and sulfur and combinations thereof.

18. The implantable medical device of claim 15 wherein said modified caprolactone is substituted at carbon 2 of Formula 5 and said substituent group comprises a branched or linear alkyl, alkenyl or alkynyl chain from C₁ to C₁₈, cyclic moieties having C₃ to C₈ including heterocycles of nitrogen, oxygen and sulfur and combinations thereof.

19. The implantable medical device of claim 15 wherein said caprolactone is substituted at carbon 3 of Formula 5 and said substituent group comprises a branched or linear alkyl, alkenyl or alkynyl chain from C₁ to C₁₈, cyclic moieties having C₃ to C₈ including heterocycles of nitrogen, oxygen and sulfur and combinations thereof.

20. The implantable medical device of claim 15 wherein said caprolactone is substituted at carbon 4 of Formula 5 and said substituent group comprises a branched or linear alkyl, alkenyl or alkynyl chain from C₁ to C₁₈, cyclic moieties having C₃ to C₈ including heterocycles of nitrogen, oxygen and sulfur and combinations thereof.

21. The implantable medical device of claim 15 wherein said caprolactone is substituted at carbon 5 of Formula 5 and said substituent group comprises a branched or linear alkyl, alkenyl or alkynyl chain from C₁ to C₁₈, cyclic moieties having C₃ to C₈ including heterocycles of nitrogen, oxygen and sulfur and combinations thereof.

22. The implantable medical device of claim 15 wherein said substitution of Formula 5 comprises a fused ring substitution.

23. The implantable medical device of claim 22 wherein said fused ring substitution comprise cyclohexane substitution at carbon 4 and 5 of Formula 5.

24. The implantable medical device of claim 22 wherein said fused ring substitution comprise cyclohexane substitution at carbon 2 and 3 of Formula 5.

25. The implantable medical device of claim 19 wherein said substitution at carbon 3 of Formula 5 comprises a tert-butyl group.

26. The implantable medical device of claim 15 wherein said second monomer is selected from the group consisting of lactide, glycolide, trimethyene carbonate, 1,8 octanediol and polyethylene glycol.

27. The implantable medical device of claim 20 wherein said substitution at carbon 4 of Formula 5 comprises a tert-butyl group.

28. The implantable medical device of claim 27 wherein the second monomer is lactide.

29. The implantable medical device of claim 15 wherein the biodegradable polymer is a terpolymer.

30. The implantable medical device of claim 15 selected from the group consisting of vascular stents, stent grafts, urethral stents, bile duct stents, catheters, guide wires, pacemaker leads, bone screws, sutures and prosthetic heart valves.

31. The implantable medical device of claim 30 which is a stent.

32. The implantable medical device of claim 31 wherein the biodegradable polymer forms a coating on the stent.

33. The implantable medical device of claim 15 wherein the polymer has a Tg of 3.9° C. to 35.9° C.

34. The implantable medical device of claim 15 wherein the drug is covalently bound to the biodegradable polymer.

35. The implantable medical device of claim 15 wherein the drug is an anti-proliferative compound.

36. An implantable medical device comprising a biodegradable polymer and a drug contained therein; wherein the biodegradable polymer comprises:
a first monomer comprising a modified caprolactone comprising a caprolactone of Formula 5 substituted at least one of carbons 1 through 5: 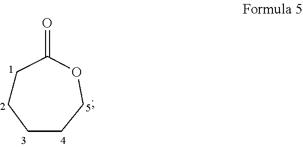
and
a second monomer selected from the group consisting of lactide, glycolide, trimethyene carbonate, 1,8 octanediol, polyethylene glycol, ε-caprolactone, and combinations thereof;
wherein the biodegradable polymer has a Tg of 3.9° C. to 35.9° C.

37. The implantable medical device of claim 36 wherein said substitution of Formula 5 comprises a tert-butyl group.

38. The implantable medical device of claim 36 which is a stent.

39. The implantable medical device of claim 38 wherein the biodegradable polymer forms a coating on the stent.

40. The implantable medical device of claim 36 wherein the drug is an anti-proliferative compound.

41. An implantable medical device comprising a biodegradable polymer and an anti-proliferative compound contained therein; wherein the biodegradable polymer comprises:
a first monomer comprising a modified caprolactone comprising a caprolactone of Formula 5 substituted at carbon 4: 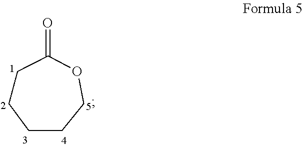
and
a second monomer selected from the group consisting of lactide, glycolide, trimethyene carbonate, 1,8 octanediol, polyethylene glycol, ε-caprolactone, and combinations thereof;
wherein the biodegradable polymer has a Tg of 3.9° C. to 35.9° C.

42. The implantable medical device of claim 41 wherein said substitution at carbon 4 of Formula 5 comprises a tert-butyl group.

43. The implantable medical device of claim 42 wherein the second monomer is lactide.

44. The implantable medical device of claim 41 which is a stent.

45. The implantable medical device of claim 44 wherein the biodegradable polymer forms a coating on the stent.
